(19) United States
(12) Patent Application Publication
(10) Pub. No.: US 2013/0131463 A1
(43) Pub. Date: May 23, 2013

(54) BODY WEIGHT MANAGEMENT DEVICE

(71) Applicants: Wataru Date, Kyoto (JP); Naoki Katsura, Shiga (JP)

(72) Inventors: Wataru Date, Kyoto (JP); Naoki Katsura, Shiga (JP)

(21) Appl. No.: 13/740,486

(22) Filed: Jan. 14, 2013

Related U.S. Application Data
(63) Continuation of application No. PCT/JP2011/064129, filed on Jun. 21, 2011.

(30) Foreign Application Priority Data
Jul. 22, 2010 (JP) 2010-165096

(51) Int. Cl. G09B 5/00; A61B 5/053

(52) U.S. Cl. CPC G09B 5/00; A61B 5/0537
USPC 600/301; 434/262

(57) ABSTRACT

A device includes a body weight obtainment unit for obtaining measurement data; a storage unit for storing the measurement data; a target calculation unit that, based on the measurement data in, calculates a target diurnal fluctuation amount including a body weight fluctuation amount during sleep and a body weight diurnal target increase/decrease amount; and a computation unit that, based on the target diurnal fluctuation amount, calculates a target value to serve as a target when measuring a pre-sleep body weight value at a second timing before the measurement subject sleeps relative to a post-waking body weight value that is measured at a first timing after the measurement subject wakes. The target calculation unit calculates the diurnal target increase/decrease amount based on the measurement data in the storage unit so that the diurnal target increase/decrease amount is within a predetermined increase/decrease percentage range of the post-waking body weight value.

### TECHNICAL FIELD

This invention relates to body weight management devices, and particularly relates to body weight management devices for managing a measurement subject's body weight that fluctuates during the day.

### BACKGROUND ART

Conventionally, a measurement subject can confirm whether his or her body weight is increasing or decreasing by measuring his or her body weight using a scale.

For example, Patent Literature 1 (JP 2003-288417A) discloses a scale that determines a state of health by displaying body weights for each day as a graph. The scale carries out the determination by comparing a target body weight to a present body weight.

Here, although it is known that body weight fluctuates, increasing from when a person wakes up to when a person goes to sleep and decreasing from when the person goes to sleep to when the person wakes up (called “diurnal fluctuation”), Patent Literature 1 does not take the diurnal fluctuation into consideration.

Accordingly, Patent Literature 2 (JP 2005-218582A) discloses a body weight management system that measures trends in changes in a body weight having taken the diurnal fluctuation into consideration. Meanwhile, Patent Literature 3 (JP 2010-14737A) discloses a system that graphs the diurnal fluctuation and determines a state of health based on the amount of change in a value that fluctuates over a week.

Patent Literature 1: JP 2003-288417A

Patent Literature 2: JP 2005-218582A

Patent Literature 3: JP 2010-14737A

### SUMMARY OF INVENTION

Recently, there has been a demand for scales to be provided with body weight management functions, such as a function for effectively supporting a diet, in addition to body weight measurement functions. In this case, a target weight loss should be set in consideration of the stated diurnal fluctuation so that the diet does not become excessive; however, although the stated Patent Literature describes systems that determine the state of health using the body weight when waking up and the body weight when going to sleep, the Patent Literature does not provide a system that takes into consideration diurnal fluctuations for each measurement subject, determines a target weight loss value for a single day that ensures the diet is not excessive, and presents that value to a measurement subject.

Therefore, one or more embodiments of the present invention provide a body weight management device for managing a body weight to achieve a target value while taking diurnal fluctuation in the body weight into consideration.

One or more embodiments of the present invention include: a body weight obtainment unit for obtaining measurement data in which a body weight measurement value for a measurement subject and a measurement day/time are associated with each other; a storage unit for storing the measurement data obtained by the body weight obtainment unit; a target calculation unit that, based on the measurement data in the storage unit, calculates a target diurnal fluctuation amount including a body weight fluctuation amount during sleep and a body weight diurnal target increase/decrease amount; and a computation unit that, based on the target diurnal fluctuation amount obtained by the target calculation unit, calculates a target value to serve as a target when measuring a pre-sleep body weight value at a second timing before the measurement subject sleeps relative to a post-waking body weight value that is measured at a first timing after the measurement subject wakes. Here, the target calculation unit calculates the diurnal target increase/decrease amount based on the measurement data in the storage unit so that the diurnal target increase/decrease amount is within a predetermined increase/decrease percentage range of the post-waking body weight value.

According to one or more embodiments of the present invention, a body weight can be managed so as to achieve a target value while taking into consideration diurnal fluctuations in the body weight.

### BRIEF DESCRIPTION OF DRAWINGS

FIG. 1 is a perspective view illustrating the external appearance of a body composition meter according to an embodiment of the present invention.

FIGS. 2A and 2B are block diagrams illustrating the configuration of the body composition meter and a server according to an embodiment of the present invention.

FIG. 3 is a diagram illustrating the functional configuration of a control unit according to an embodiment of the present invention.

FIGS. 4A through 4F are diagrams illustrating various types of data held in a storage unit according to an embodiment of the present invention.

FIGS. 5A and 5B are diagrams illustrating an outline of a procedure for calculating a target body weight according to an embodiment of the present invention.

FIG. 6 is a main flowchart illustrating operations performed by the body composition meter according to an embodiment of the present invention.

FIG. 7 is a flowchart illustrating a storage/analysis process according to an embodiment of the present invention.

FIG. 8 is a flowchart illustrating an evening target body weight calculation process according to an embodiment of the present invention.

FIG. 9 is a flowchart illustrating a morning body weight calculation process according to an embodiment of the present invention.

FIG. 10 is a flowchart illustrating a health determination process according to an embodiment of the present invention.

FIG. 11 is a diagram illustrating a degree of achievement according to an embodiment of the present invention.

FIGS. 12A through 12F are diagrams illustrating examples of display screens according to an embodiment of the present invention.

FIGS. 13A and 13B are diagrams illustrating a diurnal fluctuation in a body weight.

### DETAILED DESCRIPTION OF INVENTION

Hereinafter, an embodiment of the present invention will be described in detail with reference to the drawings. Note that in the following embodiment, identical or corresponding elements are given the same reference numerals in the drawings, and descriptions thereof will not be repeated.

First, definitions will be given for terms. In the present embodiment, “morning time” refers, with respect to body weight measurement, to a period of time spanning from, for example, 4 AM to noon (12 PM), whereas “evening time” refers to a period of time spanning from, for example, 7 PM to 2 AM. “Morning body weight” refers to a body weight measured during the morning time, whereas “evening body weight” refers to a body weight measured during the evening time. In the present embodiment, “sleeping time” refers to a time when the evening body weight is measured, whereas “waking time” refers to a time when the morning body weight is measured. In the present embodiment, it is assumed that during a single day, the body weight is measured at a timing closer to waking and at a timing closer to sleeping. To simplify the descriptions, it is assumed that the body weight (evening body weight) is measured immediately before going to bed, and that the body weight (morning body weight) is measured immediately after waking up. Note that “diurnal” refers to a single day, from the waking time to the sleeping time of a measurement subject.

“Nighttime body weight decrease” refers to a decrease in body weight caused primarily by basal metabolism, such as perspiration, occurring during the period from the sleeping time to the waking time. “Diurnal target increase/decrease amount” refers to a body weight increase/decrease amount to be used as a target during a single day. “Target diurnal fluctuation amount” refers to a fluctuation amount in the body weight composed of the “nighttime body weight decrease” and the “diurnal target increase/decrease amount”.

In the present embodiment, a body weight/body composition meter capable of obtaining not only a body weight but also a given type of body composition information, such as a body fat percentage, by measuring a body impedance, is illustrated as an example of a body weight management device, but a device that only has a function for measuring a body weight may be employed as well.

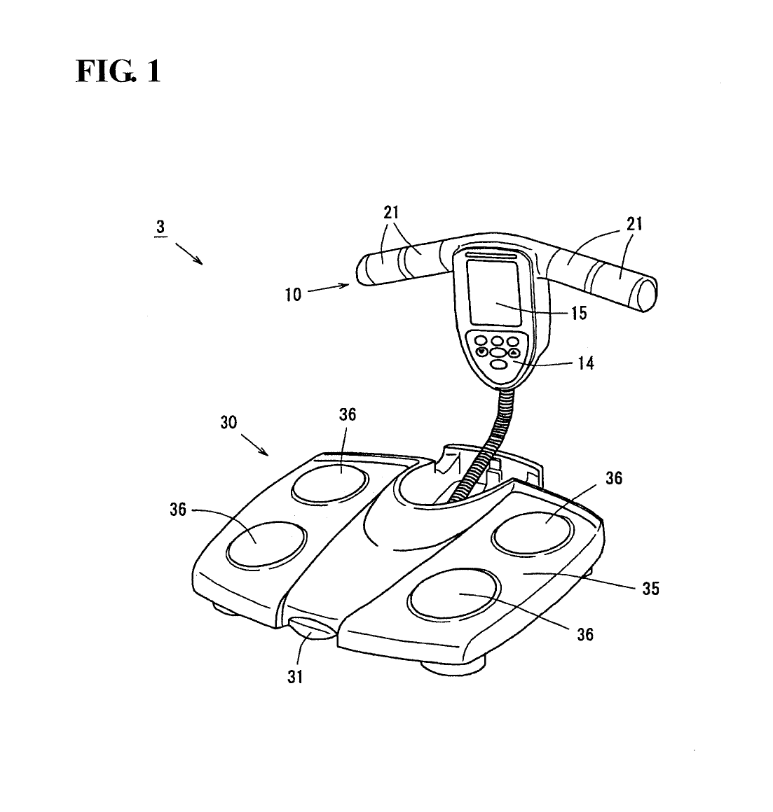

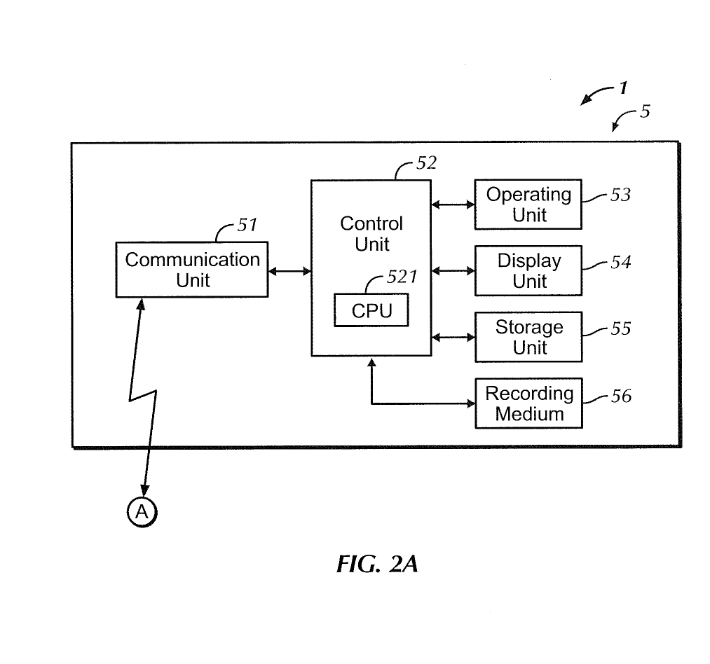

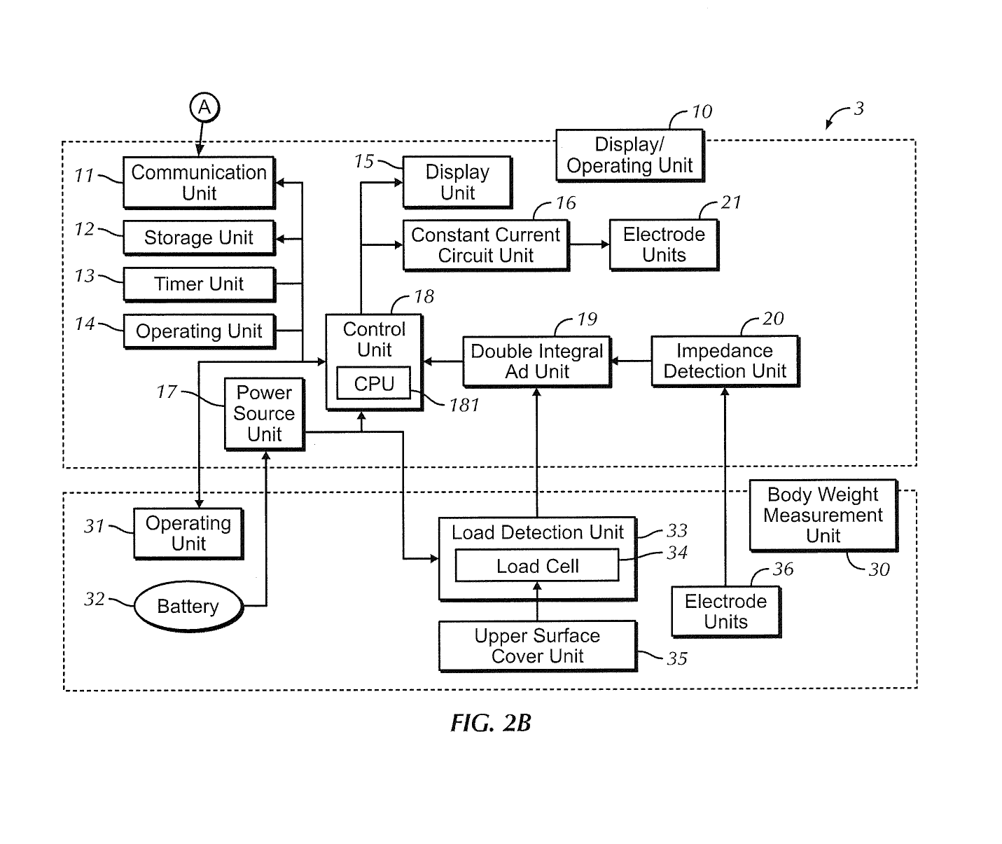

FIG. 1 illustrates the external appearance of a body weight/body composition meter 3, whereas FIGS. 2A and 2B illustrate the configuration of a body weight management system 1.

The body weight management system 1 shown in FIGS. 2A and 2B includes the body weight/body composition meter 3 and a server (server computer) 5 that communicates with the body weight/body composition meter 3. To simplify the descriptions, FIGS. 2A and 2B illustrate a single body weight/body composition meter 3 being connected to the server 5, but multiple body weight/body composition meters 3 may be connected.

In FIGS. 2A and 2B, the body weight/body composition meter 3 and the server 5 communicate wirelessly or over wires. Note that the exchange of data between the body weight/body composition meter 3 and the server 5 is not limited to communications, and the exchange may take place via a storage medium.

As shown in FIG. 1, the body weight/body composition meter 3 includes a display/operating unit 10, which is a first housing member held by a measurement subjects hand, and a body weight measurement unit 30, which is a second housing member onto which the measurement subject steps.

The display/operating unit 10 includes, as shown in FIGS. 2A and 2B, a communication unit 11, a storage unit 12, a timer unit 13, an operating unit 14, a display unit 15, a constant current circuit unit 16, a power source unit 17, a control unit 18 that includes a CPU (central processing unit) 181, a double integral AD (analog/digital) unit 19, an impedance detection unit 20, and electrode units 21.

The communication unit 11 is connected to the control unit 18, and communicates with the server 5 in accordance with a control signal from the control unit 18. Note that the communication unit 11 is not limited to communicating with the server 5; the communication unit 11 may communicate with any appropriate device, including another body information obtainment device such as a pedometer or the like, or a personal computer, mobile information terminal (a PDA (personal digital assistant), a mobile telephone, or the like), and so on.

The storage unit 12 includes an apparatus that can store information, such as a non-volatile memory, a hard disk, or the like. The storage unit 12 has information read out therefrom and written thereto in accordance with a control signal from the control unit 18, to which the storage unit 12 is connected.

The timer unit 13 is a device configured of a timer/counter that measures an amount of time from the present day/time or the like, and outputs a time to the control unit 18 as necessary.

The operating unit 14 includes multiple buttons/switches and the like (see FIG. 1) that are operated by being depressed or the like. By manipulating the operating unit 14, the measurement subject can input his/her personal information/body information, such as an ID (identification data), sex, age, height, body weight, and so on. The inputted information is outputted to the control unit 18.

The display unit 15 is configured of a display device such as a liquid-crystal display (see FIG. 1), and displays images such as text, graphics, or the like in accordance with an image signal supplied from the control unit 18.

The constant current circuit unit 16 applies a high-frequency (AC) current supplied from the power source unit 17 to current application electrode units 21 in a single direction, under the control of the control unit 18.

The power source unit 17 supplies operational electricity to the respective elements, including the control unit 18.

The control unit 18 is configured of the CPU 181 and a microcomputer (not shown) that includes a ROM (read-only memory) and a RAM (random access memory), and executes operations for controlling the respective elements, computation operations, and so on in accordance with programs and various types of data stored in the ROM or the like.

These programs and data include programs and data for body weight management.

The double integral AD unit 19 is a double integral-type AD conversion unit. During operations, the double integral AD unit 19 converts an analog signal (a voltage signal) outputted from the impedance detection unit 20 into a digital signal and outputs that digital signal to the control unit 18.

The impedance detection unit 20 detects a body impedance of the measurement subject based on a potential difference between electrode units 36 provided in the body weight measurement unit 30 and the electrode units 21 provided in the display/operating unit 10.

The electrode units 21 are provided on the surfaces of grip portions (see FIG. 1) in the display/operating unit 10, which are held in the measurement subject's hand The electrode units 21 apply the high-frequency (AC) current, supplied from the power source unit 17, to the palms of the measurement subject's hands that are gripping the grip portions.

The body weight measurement unit 30 includes an operating unit 31, a battery 32, a load detection unit 33, and the electrode units 36. The operating unit 31 functions as an input switch that is manipulated in order to switch the power on or off, and when the operating unit 31 is manipulated, an input signal is outputted to the control unit 18 in response to that manipulation.

The battery 32 supplies power to the respective elements, and in particular, to the power source unit 17.

The load detection unit 33 has multiple load cells 34 provided therein. The load detection unit 33 measures the body weight of the measurement subject that has stepped onto an upper surface cover unit 35 (see FIG. 1) that also serves as an upper surface cover of the housing member. The measured body weight is outputted to the double integral AD unit 19.

The electrode units 36 are provided in the surface of the upper surface area of the body weight measurement unit 30 (see FIG. 1) onto which the measurement subject steps, and serve as current measurement electrodes that detect a current that flows from the soles of the measurement subject's feet. The electrode units 36 include four electrodes that make contact with the left toe side, the left heel side, the right toe side, and the right heel side of the measurement subject's feet.

Each of the load cells 34 in the load detection unit 33 is disposed so as to be capable of measuring a load placed on the upper surface area of the body weight measurement unit 30, and here, are disposed below the respective electrodes in the electrode units 36. Accordingly, both the body impedance and the body weight can be measured when the measurement subject steps upon the upper surface area.

During body weight measurement, a load produced by the measurement subject's body weight is exerted on the load cells 34. Each of the load cells 34 is configured of a bending member, formed of a metal member that deforms in response to a load exerted thereon, and a strain gauge that is applied to the bending member. When the bending member bends, the strain gauge extends/contracts, and a resistance value changes in accordance with the extension/contraction of the strain gauge; the change in resistance is then derived as a load signal output. Accordingly, in the case where the measurement subject has stepped onto the upper surface area and both feet have been placed on the load cells 34, the bending member will bend due to the measurement subject's body weight that has been applied to the load cells 34, and the body weight will be measured as a change in the aforementioned load signal output.

Although the load cells 34 are used in the present embodiment as load sensors for detecting a load, it should be noted that a sensor that employs, for example, springs, a piezoelectric film, a compression element, a displacement sensor, or the like may be used as long as that element is capable of detecting the amount of a force applied to the upper surface area.

The server 5 includes a communication unit 51, a control unit 52 configured of a computer including a CPU 521, a ROM, and a RAM, an operating unit 53, a display unit 54, and a storage unit 55.

The communication unit 51 exchanges data with the body weight/body composition meter 3 under the control of the control unit 52. The CPU 521 of the control unit 52 controls the operations of the respective elements and executes various types of computations in accordance with programs and data stored in the ROM or the like.

The operating unit 53 includes a keyboard, a mouse, or the like. Signals inputted as a result of operations performed by an operator are outputted to the control unit 52.

The display unit 54 corresponds to a liquid-crystal display, a CRT (cathode ray tube) display, or the like. The display unit 54 displays images such as graphics, text, or the like in accordance with a control signal supplied from the control unit 52.

The storage unit 55 corresponds to a fixed storage device such as a hard disk, or a recording medium that can be read by the computer that includes the CPU 521, such as a CD-ROM (compact disk read-only memory), a ROM (read-only memory), a RAM (random access memory), a memory card, and so on.

The storage unit 55 stores data measured by the body weight/body composition meter 3 (body composition information, body weight data, measurement day/time data, and so on), and various types of data related to the measurement subject, including the personal information such as the measurement subject's name (ID), address, and so on.

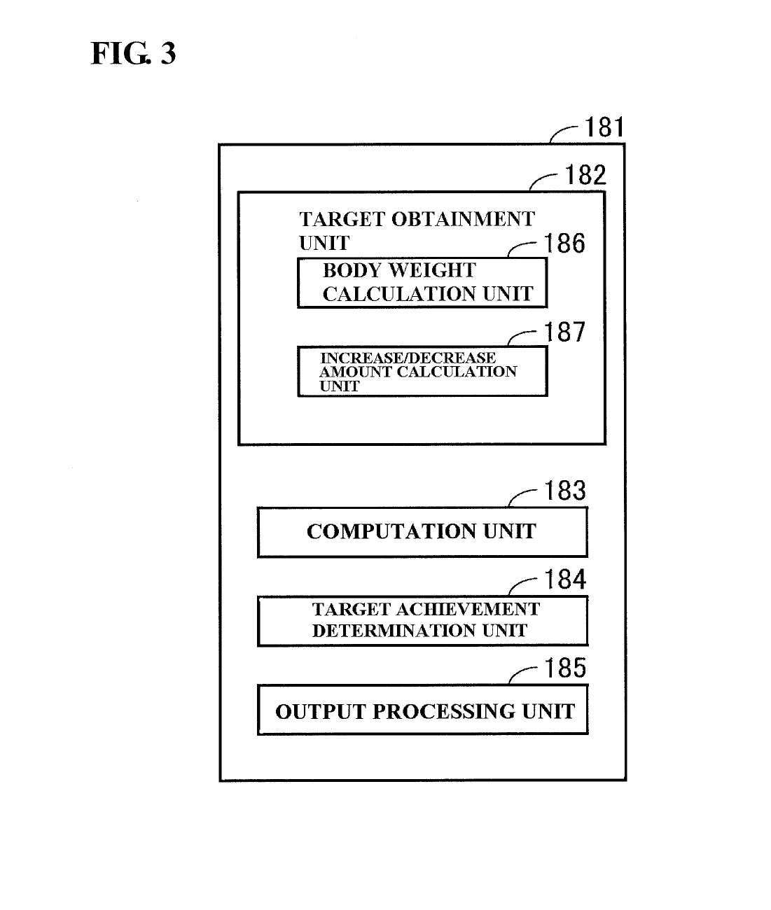

The functional configuration of the body weight/body composition meter 3, as related to body weight management, will be described with reference to FIG. 3. The CPU 181 includes a target obtainment unit 182, a computation unit 183, a target achievement determination unit 184, and an output processing unit 185 for outputting results of processing to the display unit 15. These elements are realized by programs executed by the CPU 181. These programs are stored in advance in the ROM (not shown) of the control unit 18. The functions of the respective elements are realized by the CPU 181 reading out the programs from the ROM and executing the commands in the read-out programs.

The target obtainment unit 182 obtains the target diurnal fluctuation amount for the body weight value, including the nighttime body weight decrease, which is the amount of fluctuation in the body weight value during sleep, and the diurnal target increase/decrease amount of the body weight value.

Assuming a process in which the body weight value changes in accordance with a predetermined increase/decrease amount from an initial body weight value, which is measured at an initial day/time when body weight management such as a diet has been started, to a target body weight value, which is a desired value at a target day/time when the diet has been completed, a body weight calculation unit 186 calculates (estimates) a predetermined body weight value at a predetermined day/time during that process. Here, the target day/time is a day/time at which the body weight management is complete. Meanwhile, the target body weight value is a body weight value used as a target to be achieved when the body weight management is complete.

Note that the target day/time and target body weight value are specified based on information inputted via the operating unit 53. The inputted information referred to here may be information that directly specifies the target day/time and target body weight value.

Alternatively, this information may be information that specifies information, stored in advance in the storage unit 12, for setting the target day/time and target body weight value. Specifically, it is assumed that multiple sets, each including a period from the present day/time to the target day/time and a value of the difference between a present body weight value and a target body weight value, are stored in the storage unit 12. Then, information selecting one of the multiple sets is inputted to the operating unit 53. Through this, the control unit 52 sets the target day/time and the target body weight value based on the period and the value of the difference in the selected set, the present day/time, and the present body weight value.

An increase/decrease amount calculation unit 187 compares the body weight value of the measurement subject as actually measured by the body weight measurement unit 30 on the aforementioned predetermined day/time with the aforementioned predetermined body weight value calculated (estimated) by the body weight calculation unit 186. The diurnal target increase/decrease amount is then calculated based on a result of that comparison.

Note that the increase/decrease amount calculation unit 187 may calculate the aforementioned diurnal target increase/decrease amount based on a result of comparing a difference between the target body weight value and the initial body weight value (a first difference) and a difference between the actual body weight value of the measurement subject on the aforementioned predetermined day/time and the initial body weight value (a second difference). Alternatively, the increase/decrease amount calculation unit 187 may calculate the aforementioned diurnal target increase/decrease amount based on a result of comparing an increase/decrease percentage of the target body weight value relative to the initial body weight value (a first increase/decrease percentage) with an increase/decrease percentage of the measurement subject's actual body weight value on the aforementioned predetermined day/time relative to the initial body weight value (a second increase/decrease percentage). Furthermore, the increase/decrease amount calculation unit 187 may calculate the aforementioned diurnal target increase/decrease amount using a body weight value that is from a day/time prior to the aforementioned predetermined day/time and on which measurement data has been stored as the various types of data stored in the storage unit 12, mentioned later, rather than using the aforementioned initial body weight value.

Based on the target diurnal fluctuation amount obtained by the target obtainment unit 182, the computation unit 183 calculates a diurnal target value (called an “evening target body weight” hereinafter) to serve as a target to be achieved when a pre-sleep body weight value is measured at a second timing near when the measurement subject sleeps (that is, the evening time), based on a post-waking body weight value measured at a first timing after the measurement subject has woken up (that is, the morning time).

The target achievement determination unit 184 determines whether or not the target has been achieved based on the measurement subject's pre-sleep body weight value that has actually been measured, the post-waking body weight value that has also actually been measured, and the diurnal target value calculated as described above.

The output processing unit 185 displays the measured body weight values, a result of the determination performed by the target achievement determination unit 184, and so on in the display unit 15.

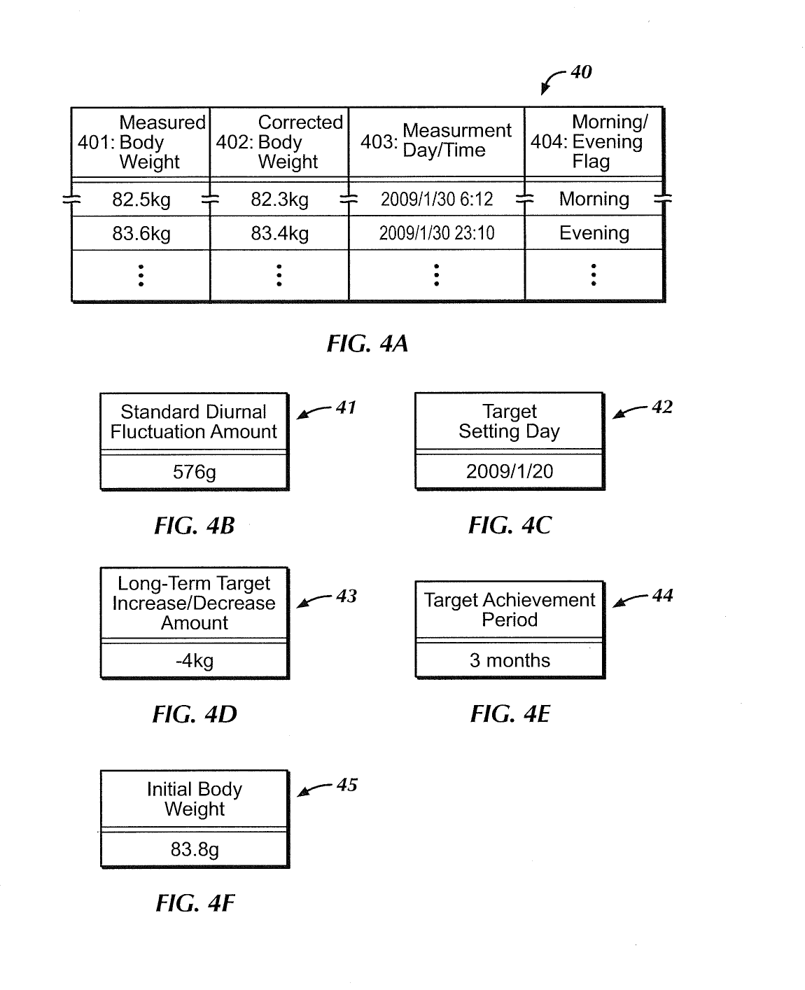

The various types of data held in the storage unit 12 will be described in detail with reference to FIG. 4. The body weight measured by the body weight measurement unit 30 is held as measurement data 40, indicated in FIG. 4A, for each measurement subject and for each measurement that is carried out. The measurement data 40 includes the following in association with each other: data 401 indicating a body weight value that has actually been measured; data 402 indicating a corrected body weight value; data 403 indicating the day/time of the body weight measurement based on timer data from the timer unit 13; and a flag 404.

The corrected body weight value indicated by the data 402 is a value resulting from correcting the body weight value in the corresponding data 401. This body weight correction will be described later.

The flag 404 indicates whether the body weight specified by the corresponding data 401 is the morning body weight measured in the morning (after waking up) or the evening body weight measured in the evening (before going to sleep).

The storage unit 12 has a capacity that enables multiple weeks' worth and multiple months' worth of measurement data 40 to be stored.

Standard diurnal fluctuation amount data 41, shown in FIG. 4B, indicates a standard diurnal fluctuation amount for the measurement subject. This standard diurnal fluctuation amount is calculated by the computation unit 183 based on past measurement data 40 (that is, one weeks' worth or two weeks' worth). This calculation may be carried out as follows.

That is, a nighttime body weight decrease indicating a fluctuation in the body weight during sleep is calculated by subtracting the next day's morning body weight from the previous evening body weight; this nighttime body weight decrease is calculated over multiple days, the average value thereof is calculated, and that average value is taken as the standard diurnal fluctuation amount.

As another method for calculating the standard diurnal fluctuation amount, it should be noted that the computation unit 183 can use a basal metabolism amount (Kcal) found from the measurement subject's body impedance as calculated by the impedance detection unit 20. Note that the method for calculating the basal metabolism amount employs a known procedure. Generally, 9 Kcal is required to burn 1 g of fat (however, because human fat contains 20% water, this number is approximately 7 Kcal), 4 Kcal is required to burn 1 g of carbohydrate, and 4 Kcal is required to burn 1 g of protein; accordingly, the computation unit 183 can calculate the nighttime body weight decrease amount based on these consumed Kcal amounts, the calculated basal metabolism amount, and a sleep amount (the total amount of time from the sleeping time to the waking time). This nighttime body weight decrease is calculated over multiple days, the average value thereof is calculated, and that average value can then be taken as the standard diurnal fluctuation amount.

Note that the standard diurnal fluctuation amount is not limited to being calculated for each measurement subject; a fixed value can be set for the diurnal fluctuation amount of a typical person set in advance.

Target setting day data 42 is indicated in FIG. 4C. The target setting day data 42 indicates the date at which the measurement subject started body weight management, such as a diet, using the body weight/body composition meter 3. In other words, this data indicates the date at which the measurement subject set (inputted) the data shown in FIGS. 4A through 4F for the purpose of body weight management.

Long-term target increase/decrease amount data 43, shown in FIG. 4D, indicates a target value for a body weight increase/decrease amount, inputted by the measurement subject manipulating the operating unit 14. For example, a negative value is inputted in the case where the measurement subject wishes to lose weight through a diet, 0 is inputted in the case where the measurement subject wishes to maintain his/her body weight, and a positive value is inputted in the case where the measurement subject wishes to gain weight. Note that this value may be a target body weight value instead of an increase/decrease amount.

Target achievement period data 44, shown in FIG. 4E, holds a target achievement period set by the measurement subject manipulating the operating unit 14. The target achievement period indicates a period by the end of which the long-term target increase/decrease amount is to be achieved.

When this target achievement period is inputted and set, the target obtainment unit 182 determines, as appropriate, whether or not a diurnal increase/decrease amount that serves as a daily norm, obtained by dividing the long-term target increase/decrease amount by the number of days of the target achievement period, falls within a predetermined range. In the case where it is determined that the amount is outside of the predetermined range, an error display is made in the display unit 15. The measurement subject is then prompted to re-input the target achievement period until the amount is determined to fall within the predetermined range. Through this, an excessive diet that places a burden on the measurement subject's body or the like can be avoided.

With respect to body weight fluctuations, experiments performed by the inventors indicated that for a healthy adult, a weight loss (or gain) for one month that is an increase/decrease percentage of the present body weight within the predetermined range, or in other words, within 2% to 10%, will not be unhealthy. Accordingly, the present embodiment is set so that the decrease amount (or increase amount) over one month is an amount that is 2% to 10% of the present body weight.

Initial body weight data 45, shown in FIG. 4F, indicates a body weight measured on the initial day/time when the body weight management such as a diet was started, or in other words, indicates the body weight measured on the date specified by the target setting day data 42.

Through this, a body weight obtained by adding the long-term target increase/decrease amount data 43 to the initial body weight data 45 corresponds to the target body weight at a target date when the body weight management, such as the diet, is complete (that is, a date obtained by adding the target achievement period data 44 to the target setting day data 42).

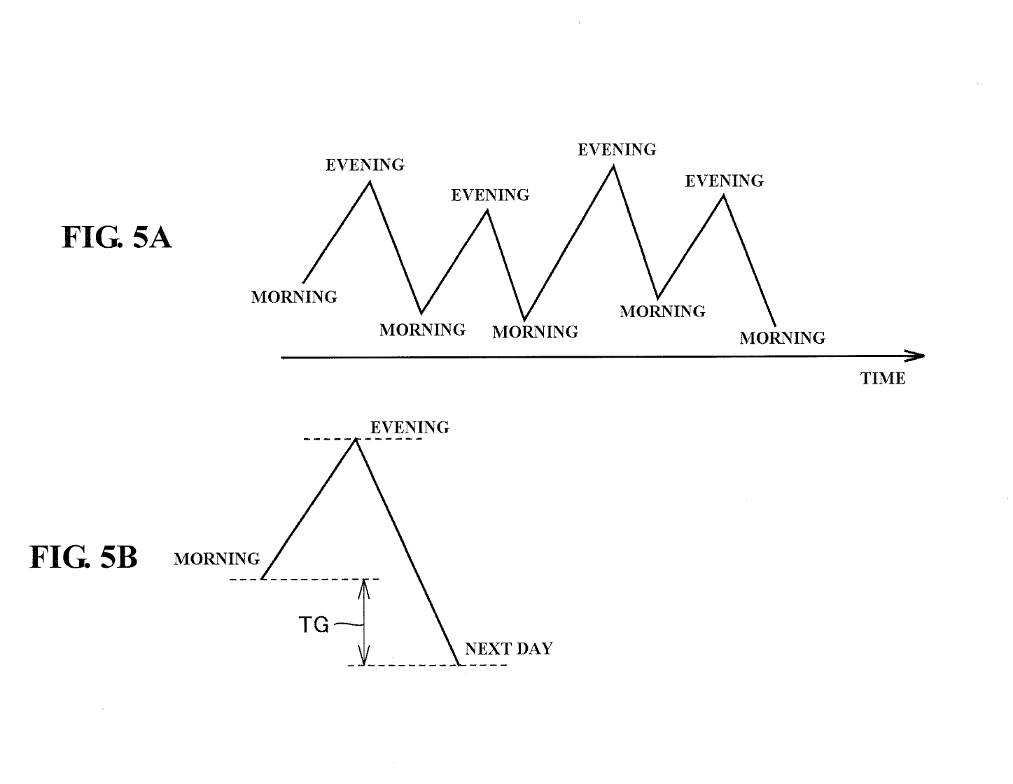

Here, a method for calculating the target body weight in a single day, or in other words, the evening body weight, in the case where body weight management for the purpose of dieting (that is, losing weight) is carried out, will be described with reference to FIGS. 5A and 5B.

As shown in FIG. 5A, generally, the measurement subject's morning body weight and evening body weight fluctuate during a single day. Looking at the diurnal fluctuations shown in FIG. 5A, the morning body weight is a body weight measured after waking up and can thus be taken as the minimum body weight for that day; thus, as shown in FIG. 5B, the difference between the morning body weight for that day and the morning body weight for the next day can be determined as the amount that should be adjusted, or in other words, as a target weight loss TG for a single day. Accordingly, the evening body weight for that day can be calculated by adding the target weight loss TG for a single day to the morning body weight of that day.

In the present embodiment, the target weight loss for a single day is taken as the target diurnal fluctuation amount, and the target diurnal fluctuation amount is calculated from the “nighttime body weight decrease” and the “diurnal target increase/decrease amount”.

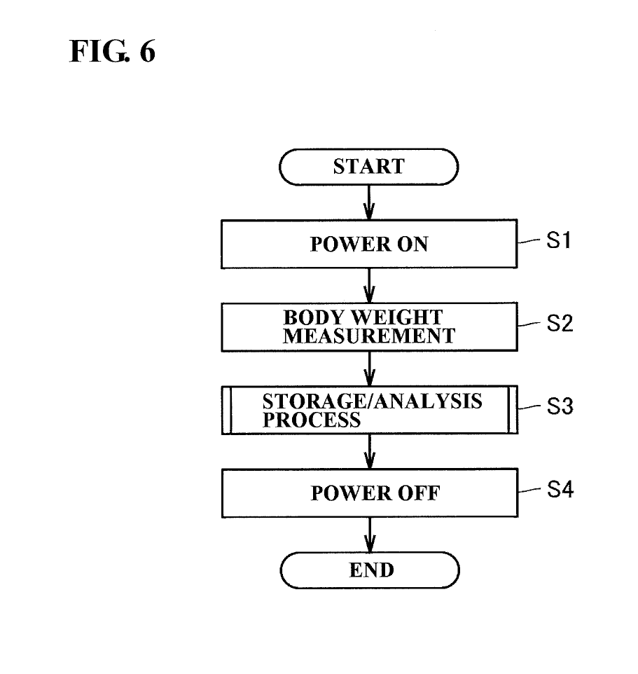

FIGS. 6 through 10 are flowcharts illustrating operations executed by the CPU 181 in the control unit 18 of the body weight/body composition meter 3. Here, processes for executing body weight management for the purpose of dieting will be described based on these flowcharts. These flowcharts are held in a memory in the control unit 18 or the storage unit 12 in advance as programs, and the processes are realized by the CPU 181 reading out the programs and executing the commands contained therein.

Note that the data 41 through data 45 shown in FIG. 4 is assumed to be held in the storage unit 12 in advance. In addition, it is assumed that nine days' worth of morning body weight and evening body weight measurement data 40 from the date specified in the target setting day data 42 is held in the storage unit 12. Thus descriptions will be given assuming that the body weight measurement for the tenth day is being carried out here.

As shown in FIG. 6, the CPU 181 starts up in response to the measurement subject inputting a power on instruction through the operating unit 14 (step S1), and, using the load detection unit 33, measures the body weight of the measurement subject who has stepped onto the upper surface cover unit 35 (see FIG. 1) (step S2). At this time, the CPU 181 calculates the body composition information based on the body impedance detected by the impedance detection unit 20 (see FIGS. 2A and 2B) using the electrode units 36 of the body weight measurement unit 30 and the electrode units 21 of the display/operating unit 10.

After this, the CPU 181 executes a storage/analysis process (step S3), turns the power off (step S4), and ends the processing.

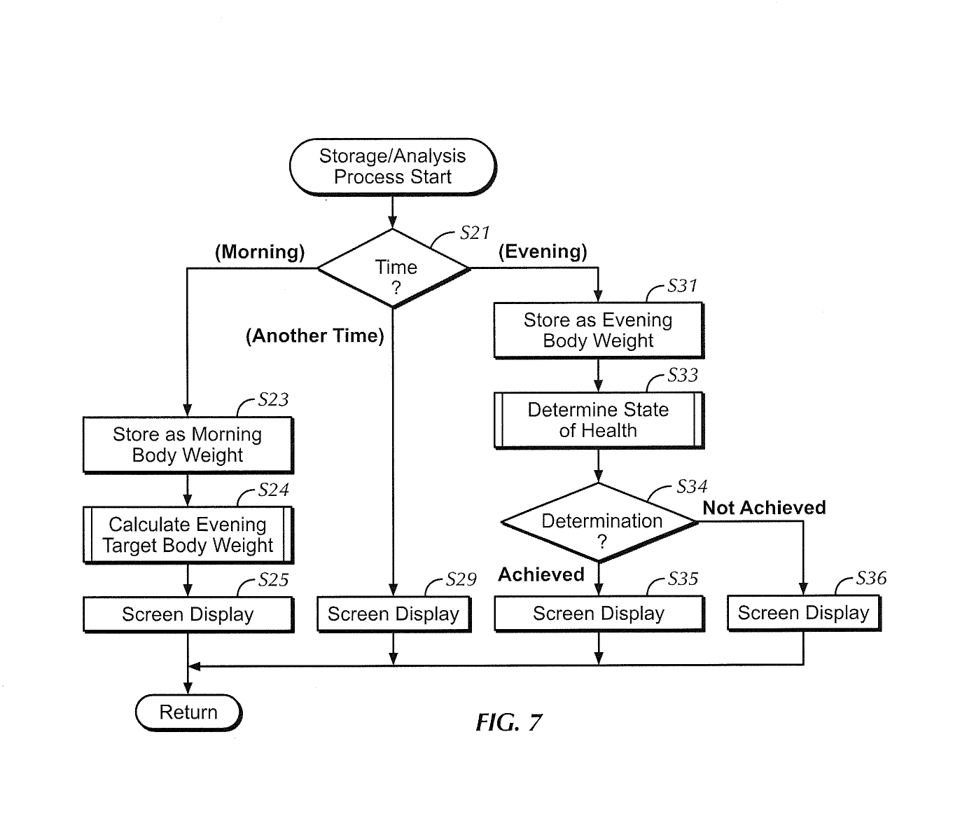

FIG. 7 is a flowchart illustrating the storage/analysis process (see step S3 in FIG. 6). As shown in FIG. 7, the CPU 181 obtains data of the present day/time, which is the day/time of the body weight measurement in the aforementioned step S2, from the timer unit 13, and determines whether the present day/time corresponds to the morning time, the evening time, or another time (step S21). It is assumed that data of the morning time and the evening time is held in advance in a memory within the control unit 18. The determination can be made by comparing the morning time and evening time data read out from the memory with the present day/time data.

If it is determined to be morning time (“morning” in step S21), the CPU 181 generates the measurement data 40 using the body weight measured in step S2 as the morning body weight, and stores the generated measurement data 40 in the storage unit 12. This measurement data 40 includes the data 401 indicating the measured body weight, the data 403 indicating the present day/time, and the flag 404 indicating “morning”.

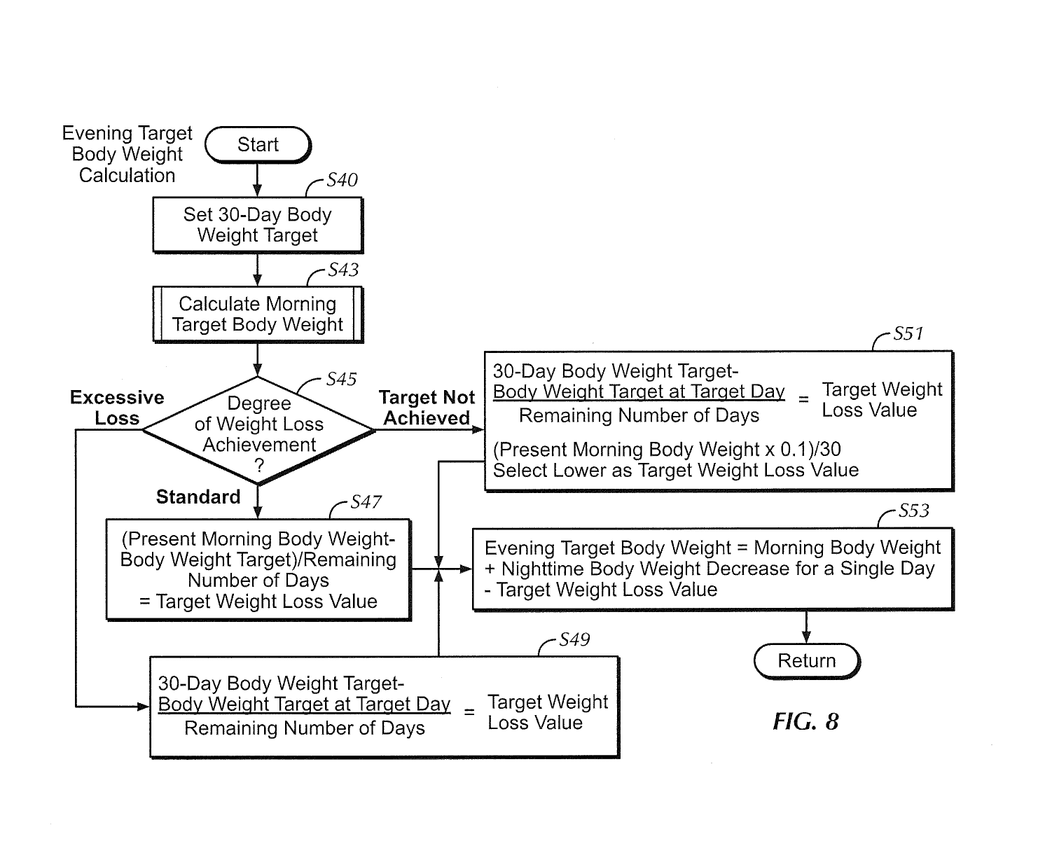

When the measurement data 40 of the morning body weight has been stored, the evening body weight to serve as the target, or in other words, the diurnal target value, is calculated (step S24). This process will be described later with reference to FIG. 8.

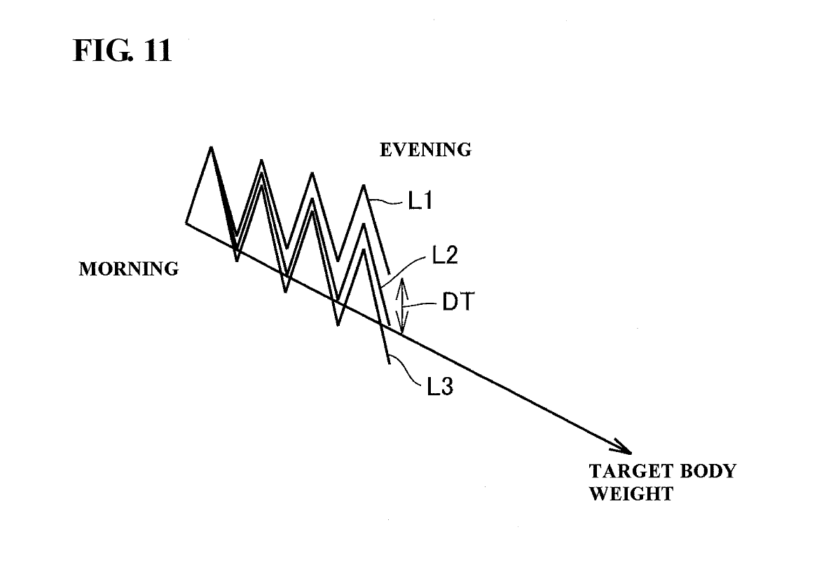

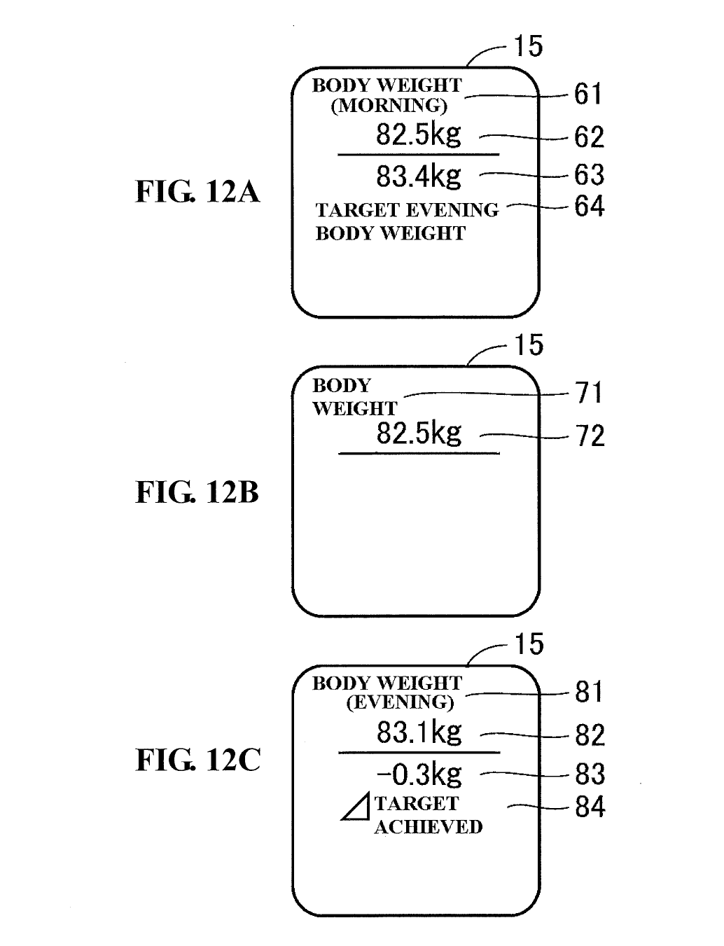

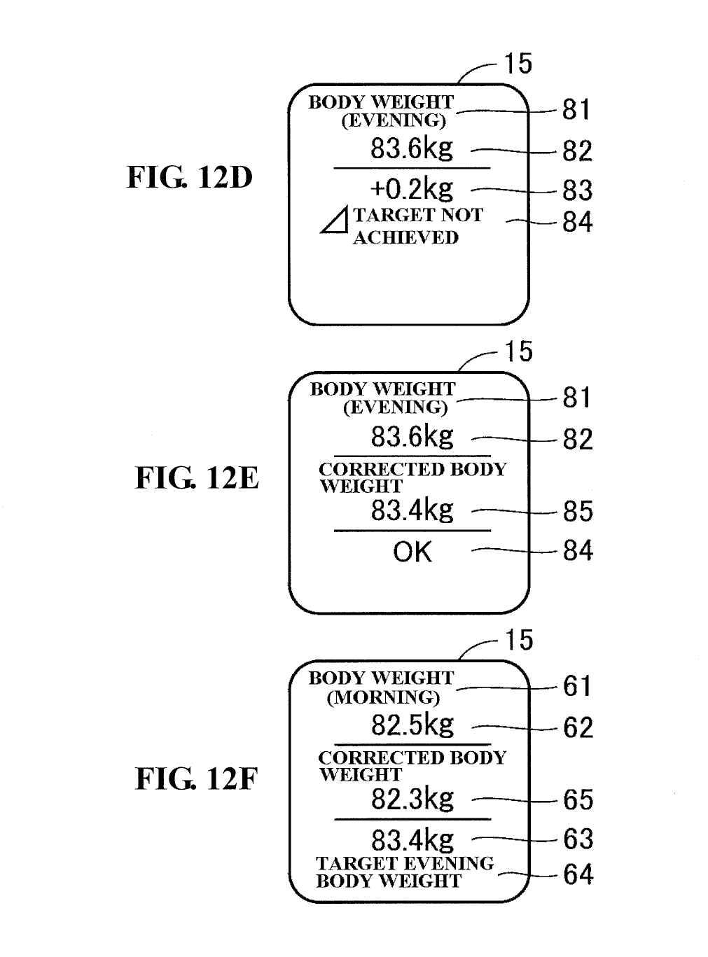

When the diurnal target value has been calculated, the output processing unit 185 displays, in the display unit 15, a screen (see FIG. 12A) including the following display items: a target for the evening body weight indicated by the calculated diurnal target value, and the body weight measured in step S2 (step S25).

The screen in FIG. 12A includes, as display items, a morning body weight title display 61 indicating that the display concerns the morning body weight, a measured body weight display 62 indicating the measured value for the morning body weight, a target display 63 indicating the target value of the evening body weight, which is the calculated diurnal target value, and a target title display 64 indicating that the value is the target value for the evening body weight. Note that the screen shown in FIG. 12F may be displayed instead of the screen shown in FIG. 12A. In the screen shown in FIG. 12F, a corrected morning body weight display 65 has been added to the display items in FIG. 12A. This corrected morning body weight will be described later.

In the case where the evening time has been determined in step S21 (“evening” in step S21), the CPU 181 generates the measurement data 40 using the body weight obtained in step S2 as the evening body weight, and stores the generated measurement data 40 in the storage unit 12 (step S31). This measurement data 40 includes the data 401 indicating the measured body weight, the data 403 indicating the present day/time, and the flag 404 indicating “evening”.

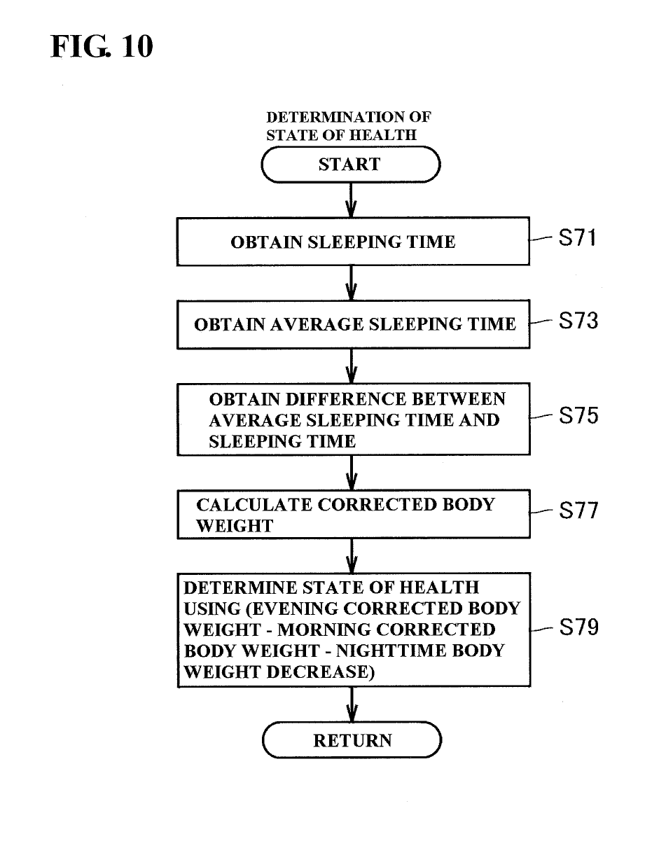

When the measurement data 40 of the evening body weight is stored, the CPU 181 determines a degree to which the target has been achieved based on the measurement data 40 in the storage unit 12 (step S33). This determination will be called a “health determination” hereinafter. This determination will be described later with reference to FIG. 10.

In the case where the result of the determination indicates that the target has been achieved (“achieved” in step S34), the output processing unit 185 displays the screen indicated in FIG. 12C in the display unit 15 (step S35).

The screen in FIG. 12C includes, as display items, an evening body weight title display 81 indicating that the display concerns the evening body weight, a measured body weight display 82 indicating the measured value of the evening body weight, a difference display 83 indicating the difference between the diurnal target value (that is, the target for the evening body weight) and the evening body weight that has actually been measured, and a target achievement result display 84 indicating whether or not the target has been achieved. In this example, the difference display 83 and the target achievement result display 84 indicate that the measured evening body weight is 0.3 kg lighter than the diurnal target value and that the target has been achieved.

In the case for the target has not been achieved (“not achieved” in step S34), the output processing unit 185 displays the screen indicated in FIG. 12D in the display unit 15 (step S36). Because the screen shown in FIG. 12D displays the same items as those displayed in the screen shown in FIG. 12C, the same reference numerals will be assigned to identical items and detailed descriptions thereof will be omitted. In this example, the difference display 83 and the target achievement result display 84 indicate that the measured evening body weight is 0.2 kg heavier than the diurnal target value and that the target has not been achieved.

Returning to step S21, if it is determined that the time corresponds to neither the morning time nor the evening time (“another” in step S21), the measured body weight data is stored in the storage unit 12 in association with the measurement time, and the output processing unit 185 displays the screen shown in FIG. 12B in the display unit 15 (step S29).

The screen in FIG. 12B includes, as display items, a measured body weight title display 71 indicating that the measured body weight is being displayed, and a measured body weight display 72 that displays the measured body weight.

After the processes of steps S25, S29, S35, and S36, the processing returns to the original processing in FIG. 6.

Evening Target Body Weight Calculation Process

An evening target body weight calculation process (step S24 in FIG. 7) will be described with reference to FIG. 8.

First, the body weight calculation unit 186 sets a body weight target to be achieved by a predetermined target day following the date (initial day) indicated in the target setting day data 42 (for example, 30 days after the initial day) (step S40). Here, a value is calculated within a range that ensures the diet is not excessive, so that, for example, the body weight 30 days after the initial day is 2% lower than the present body weight. This 2% reduction in body weight will be referred to here as a “30-day body weight target”. Although 2% is a value set in advance in a memory of the body weight/body composition meter 3, the configuration may be such that the user can vary the set value.

Although the target day is described in step S40 as being 30 days after the initial day, it should be noted that the target day is not limited to 30 days, and may be set as appropriate within the period indicated by the target achievement period data 44. In addition, the calculation of the evening target body weight is assumed here to be carried out from, for example, the tenth day following the initial day; this makes it possible to use multiple days' worth of the measurement data 40 obtained from body weight measurements starting on the initial day in order to calculate the evening body target body weight.

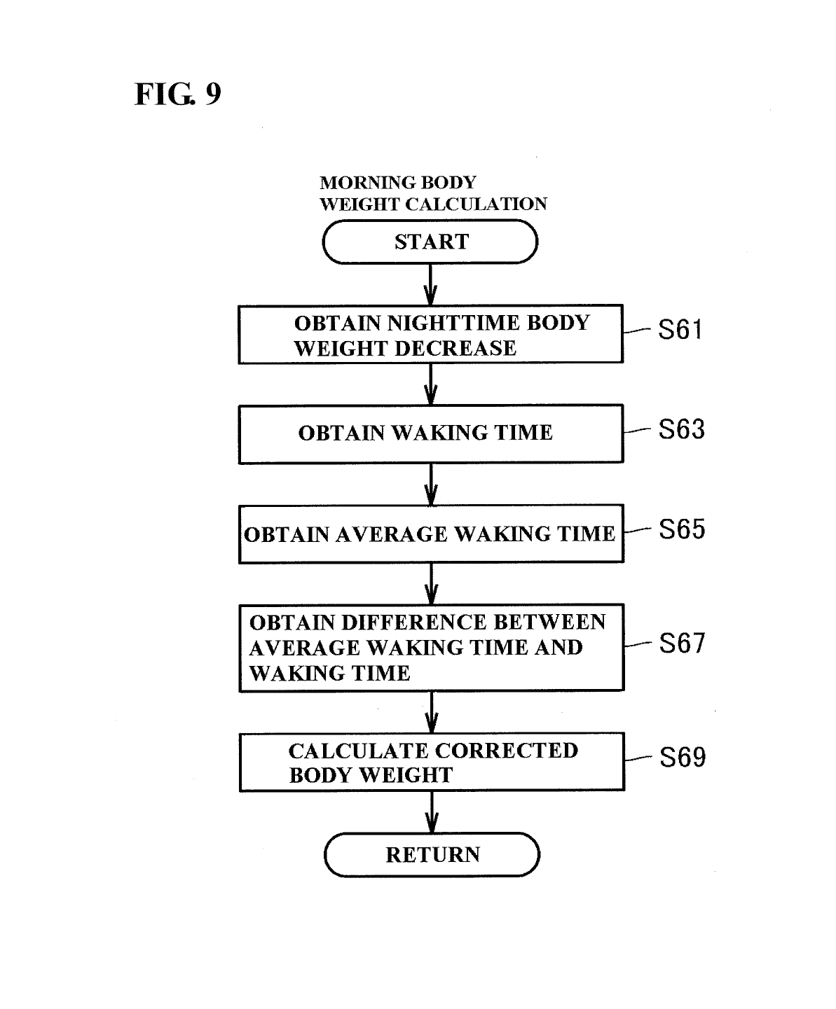

When the 30-day body weight target has been calculated, next, a corrected morning body weight is calculated using the measured body weight (step S43). In other words, the morning body weight indicated by the data 401 in the measurement data 40 stored in the storage unit 12 in step S23 is corrected based on the standard diurnal fluctuation amount data 41 and the morning time indicated by the data 402 in that measurement data 40. The corrected morning body weight is then held in the measurement data 40 as the data 402. This correction process will be described later with reference to FIG. 9.

A degree of weight loss achievement is then determined using the calculated corrected morning body weight (step S45). An outline of this determination will be described with reference to FIG. 11.

The straight line arrow moving downward to the right in FIG. 11 expresses the process of the body weight value changing according to the stated 2% decrease from the initial day to 30 days thereafter. The increase/decrease amount calculation unit 187 calculates the body weight on the tenth day indicated by the straight line arrow. Then, the morning body weight actually measured on the tenth day is compared with the body weight calculated on the tenth day indicated by the straight line arrow. The determination is made based on the result of this comparison.

Specifically, in the case where the morning/evening body weight measured from the initial day changes as indicated by the case L2, or in other words, in the case where the slope of a straight line that connects the morning body weights in the case L2 (that is, the body weight increase/decrease percentage) is approximately the same as the slope of the straight line arrow (the body weight increase/decrease percentage), a determination of “standard” is made. To be more specific, in the case where both the morning body weight measured on the tenth day and the body weight calculated on the tenth day as indicated by the straight line arrow are equal, the weight loss is considered to be proceeding normally, and thus the determination of “standard” is made.

However, in the case where the result of the comparison indicates that the morning/evening body weight measured from the initial day changes as indicated by the case L1, or in other words, in the case where the slope of a straight line that connects the morning body weights in the case L1 (that is, the body weight increase/decrease percentage) is greater than the slope of the straight line arrow (the body weight increase/decrease percentage), a determination of “target not achieved” is made. To be more specific, in the case where the morning body weight measured on the tenth day is greater than the body weight calculated on the tenth day as indicated by the straight line arrow, a determination of “target not achieved”, indicating that the weight loss target is not being achieved, is made.

Furthermore, in the case where the result of the comparison indicates that the morning/evening body weight measured from the initial day changes as indicated by the case L3, or in other words, in the case where the slope of a straight line that connects the morning body weights in the case L3 (that is, the body weight increase/decrease percentage) is lower than the slope of the straight line arrow (the body weight increase/decrease percentage), a determination of “excessive loss” is made. To be more specific, in the case where the morning body weight measured on the tenth day is lower than the body weight calculated on the tenth day as indicated by the straight line arrow, a determination of “excessive loss”, indicating that the weight loss is proceeding at a higher rate than the target weight loss, is made.

In this manner, the increase/decrease amount calculation unit 187 determines the degree of achievement for the weight loss based on the difference in the aforementioned slopes (the body weight increase/decrease percentages), and more specifically, based on a difference DT between the morning body weight measured on the tenth day and the body weight calculated on the tenth day as indicated by the straight line arrow.

Returning to the process in FIG. 8, in the case where “excessive loss” has been determined (“excessive loss” in step S45), the increase/decrease amount calculation unit 187 calculates the target weight loss value (that is, the diurnal target increase/decrease amount) using Formula 1 (step S49).

(30-day body weight target−body weight target for target day/remaining number of days) Formula 1

In the case where a determination of “standard” has been made, the increase/decrease amount calculation unit 187 calculates the target weight loss value using Formula 2, or (present morning body weight−body weight target for target day/remaining number of days) (step S47).

In the case where a determination of “target not achieved” has been made, the increase/decrease amount calculation unit 187 calculates the target weight loss value using the aforementioned Formula 1 and Formula 3, shown below, and sets the lower of the value calculated through Formula 1 and the value calculated through Formula 3 as the target weight loss value (step S51).

(present morning body weight×0.1)/30 days Formula 3

With respect to Formula 1 through Formula 3, the “body weight target for target day” is calculated by adding the value of the long-term target increase/decrease amount data 43 to the body weight indicated in the initial body weight data 45. Meanwhile, the “remaining number of days” is calculated by subtracting ten days (the number of days that has passed since the initial day) from the number of days indicated in the target achievement period data 44. Furthermore, the “present morning body weight” indicates the morning body weight measured in step S2 or the corrected morning body weight calculation in step S43.

When the target weight loss value has been calculated, the computation unit 183 calculates the evening target body weight, which is the diurnal target value (step S53). In other words, the computation unit 183 calculates the evening target body weight using the following Formula 4.

evening target body weight=morning body weight+nighttime body weight decrease in single day−target weight loss value Formula 4

Note that in Formula 4, the “morning body weight” is the morning body weight measured in step S2 or the corrected body weight calculated in step S43, the “nighttime body weight decrease in single day” indicates the value of the standard diurnal fluctuation amount data 41, and the “target weight loss value” indicates the value calculated in step S47, step S49, or step S51.

After the evening target body weight has been calculated using Formula 4, the procedure returns to the original process in FIG. 7.

As described using FIG. 8 and FIG. 11, when body weight management such as a diet is being carried out, the evening target body weight can be variably determined in accordance with the degree to which the diet is being achieved (see FIG. 11) by using Formula 1 through Formula 4.

Specifically, in the case where the degree of achievement has been determined as “excessive loss”, a target weight loss value in which the body weight value changes in accordance with the original 2% decrease is determined using Formula 1, so that the diet does not become excessive. Meanwhile, in the case where a determination of “standard” has been made, a target weight loss value in which the body weight value changes in accordance with the original 2% decrease is determined using Formula 2, so that a proper degree of weight loss is maintained. On the other hand, in the case where a determination of “target not achieved” has been made, the lower of the value calculated through Formula 1 and the value calculated through Formula 3 is selected, thus bringing the target weight loss value into a predetermined range. In other words, Formula 3 specifies a formula for calculating a target weight loss value for a 10% loss from the present body weight over 30 days (one month). The target weight loss value based on Formula 3 corresponds to an upper limit value (a 10% weight loss) that, as mentioned above, will not negatively impact the health, as shown through experiments carried out by the inventors. Accordingly, in the present embodiment, although the target weight loss value is determined so that the target will be achieved, that value is also determined so as to fall within an appropriate range that does not exceed the stated upper limit value (that is, a 2% loss to a 10% loss).

Morning Body Weight Calculation Process

The process through which the computation unit 183 calculates the morning body weight (step S43 in FIG. 8) will be described with reference to the flowchart shown in FIG. 9.

First, the computation unit 183 obtains the nighttime body weight decrease. This nighttime body weight decrease is specified in the standard diurnal fluctuation amount data 41 read out from the storage unit 12 (step S61). The nighttime body weight decrease may, as mentioned earlier, be estimated based on the measurement subject's basal metabolism amount.

Next, the computation unit 183 obtains the waking time (morning time) using the timer unit 13 (step S63). An average waking time is then obtained (step S65). Specifically, of multiple days' worth of past measurement data 40 stored in the storage unit 12, the average value of waking times indicated by the data 403 in the measurement data 40 whose flag 404 indicates “morning” is calculated in order to obtain the average waking time.

Next, the computation unit 183 calculates a difference between the waking time obtained in step S63 and the average waking time calculated in step S65 (step S67). Then, based on the calculated difference in the times, the morning body weight indicated by the data 401 in the measurement data 40 measured in step S2 and stored in the storage unit 12 is corrected. The corrected morning body weight (morning corrected body weight) is then held in that measurement data 40 as the data 402 (step S69).

In the correction, if, for example, the nighttime body weight decrease is 576 g (see FIG. 4B), a value obtained by dividing the nighttime body weight decrease by an average sleep time (that is, the total time from the sleeping time to the waking time) is multiplied by the time difference obtained in step S67, and the result of that multiplication is added to the morning body weight indicated by the data 401 in the measurement data 40 presently measured and obtained. In other words, the morning body weight is corrected so as to increase by an amount equal to the value of the multiplication result if the time difference is negative (that is, if the obtained waking time is earlier than the average waking time), and to decrease by an amount equal to the value of the multiplication result if the time difference is positive (that is, if the obtained waking time is later than the average waking time).

After this, the procedure returns to the process of FIG. 8.

Health Determination Process

A health determination process (step S33 in FIG. 7) performed by the target achievement determination unit 184 will be described with reference to FIG. 11.

First, the target achievement determination unit 184 obtains the sleeping time (evening time) using the timer unit 13 (step S71), and obtains an average sleeping time (step S73). Specifically, of multiple days' worth of past measurement data 40 stored in the storage unit 12, the average value of sleeping time indicated by the data 403 in the measurement data 40 whose flag 404 indicates “evening” is calculated in order to obtain the average sleeping time.

Next, the target achievement determination unit 184 calculates a difference between the sleeping time obtained in step S71 and the average sleeping time calculated in step S73 (step S75). Then, based on the calculated difference in the times, the evening body weight indicated by the data 401 in the measurement data 40 measured in step S2 and stored in the storage unit 12 is corrected. The corrected evening body weight (evening corrected body weight) is then held in that measurement data 40 as the data 402 (step S77).

In the correction, if, for example, the nighttime body weight decrease is 576 g (see FIG. 4B), a value obtained by dividing the nighttime body weight decrease by an average sleep time (that is, the total time from the sleeping time to the waking time) is multiplied by the time difference obtained in step S75, and the result of that multiplication is added to the evening body weight indicated by the data 401 in the measurement data 40 presently measured and obtained. In other words, the evening body weight is corrected so as to increase by an amount equal to the value of the multiplication result if the time difference is negative (that is, if the obtained sleeping time is earlier than the average sleeping time), and to decrease by an amount equal to the value of the multiplication result if the time difference is positive (that is, if the obtained sleeping time is later than the average sleeping time).

Next, in step S79, the target achievement determination unit 184 calculates a value based on the formula (evening corrected body weight−morning corrected body weight−nighttime body weight decrease). Here, the evening corrected body weight is the value calculated in step S77, whereas the morning corrected body weight indicates the corrected morning body weight indicated by the data 402 of the measurement data 40 stored in the storage unit 12 that includes the data 403 indicating the previous measurement day/time. The nighttime body weight decrease indicates the standard diurnal fluctuation amount data 41 shown in FIG. 4B.

The target achievement determination unit 184 determines, if the value resulting from the calculation is 0 or a negative value, that the body weight is not increasing and is maintaining its present state, or in other words, that the state of health is “OK” (that is, that the target is being achieved). On the other hand, if the value is a positive value, it is determined that the body weight is increasing, or in other words, that the state of health is “NG” (that is, that the target is not being achieved). Through this, a body weight increase/decrease can be determined, and the result of this determination can be outputted as a determination regarding the state of health.

Furthermore, the target achievement determination unit 184 may carry out the determination as follows. In other words, the aforementioned formula is changed to (evening corrected body weight—morning corrected body weight—nighttime body weight decrease—target weight loss value). Here, the target weight loss value refers to the value calculated in step S47, S49, or S51 in FIG. 8.

The target achievement determination unit 184 determines, if the value resulting from the calculation carried out through the changed formula is 0 or a negative value, that the weight loss target is being achieved, or “OK” (target achieved), and determines, if the value is a positive value, that the body weight is increasing, or “NG” (target not achieved). Through this, it can be determined whether or not the weight loss amount indicated by the target weight loss value has been achieved, and the result of that determination can then be outputted.

The display screen for the case where the target achievement determination unit 184 has determined that the target has been achieved is not limited to that shown in FIG. 12C, and may be that shown in FIG. 12E. In the screen shown in FIG. 12E, an evening corrected body weight display 85 has been added to the display items of FIG. 12C, and the display 84 has been changed to the text “OK”. Meanwhile, in the display screen for the case where it has been determined that the target is not achieved, the display 84 in FIG. 12E is “NG”.

In this manner, by using the standard diurnal fluctuation amount data 41 (the basal metabolism amount during sleep) and the sleep time (the total time from the sleeping time to the waking time), the morning body weight or evening body weight can be corrected using a body weight increase/decrease amount based on differences in the waking time, sleeping time, and so on in a single day, and a more accurate determination of the measurement subject's state of health can be made based on the rhythm of his/her lifestyle activity times by using the corrected body weights.

Furthermore, by using the corrected morning body weight as the morning body weight in Formula 4, the evening target body weight can be calculated based on the rhythm of the lifestyle activity times.

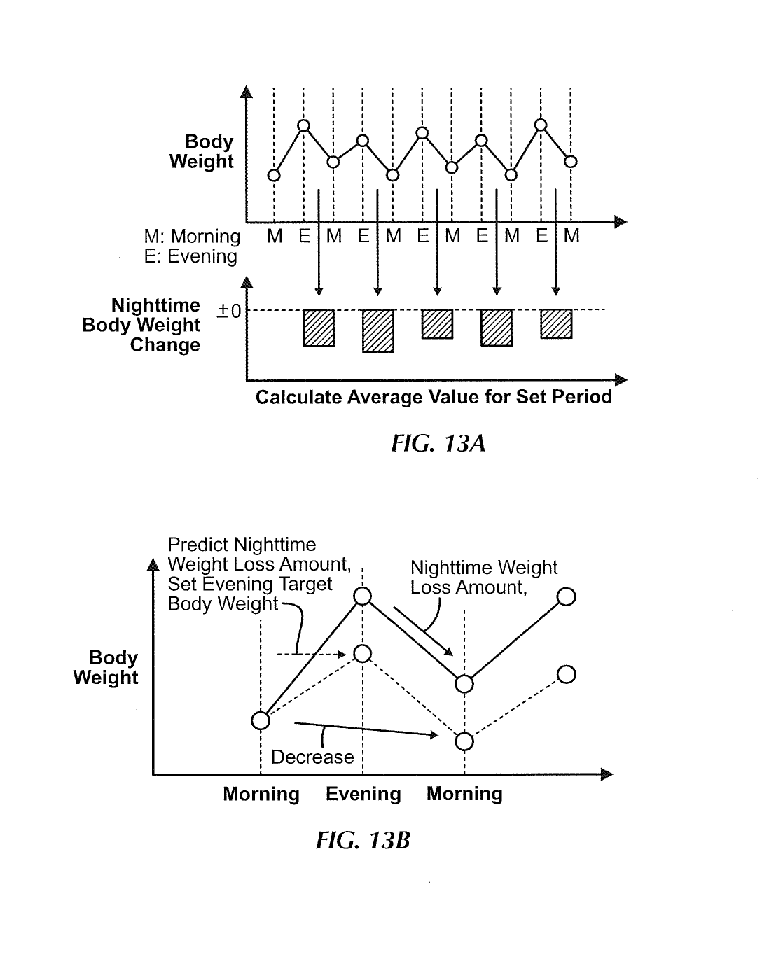

In the present embodiment, the standard diurnal fluctuation amount data 41 can be accurately set on a measurement subject-by-measurement subject basis. In other words, as shown in FIG. 13A, a body weight fluctuation amount during sleep is calculated by subtracting the measurement subject's morning body weight from the evening body weight, and by calculating an average value for multiple days' worth of the body weight fluctuation amount during sleep, the standard diurnal fluctuation amount data 41 can be determined based on the measurement subject's actual body weight increase/decrease amount.

Furthermore, by using this calculation method, the correct standard diurnal fluctuation amount can be determined regardless of whether the body weight is in an increasing trend or a decreasing trend.

Furthermore, in the present embodiment, the tendency for a person's body weight to increase from morning to evening and decrease from evening to morning (see FIG. 5A and FIG. 13B) is taken into consideration using Formula 4, and by calculating and outputting the evening target body weight, which is a guideline for the degree to which the increase from the morning time to the evening time should be suppressed, the measurement subject can obtain support information for managing increases/decreases in his/her body weight on a daily basis.

Through the aforementioned configurations and operations, the measurement subject can confirm the evening target body weight, indicating the degree to which the diurnal body weight fluctuation should be controlled, and can manage his/her diurnal body weight fluctuations in a deliberate manner so as to achieve the target. Accordingly, when dieting, for example, the measurement subject can obtain support information for determining whether or not the diet is progressing properly on a daily basis, which is an extremely short period of time.

Variations

Although the body weight/body composition meter 3 performs all the computations in the aforementioned embodiment, it should be noted that the configuration may be such that the server 5 carries out various types of computations, outputs, and so on.

In this case, the configuration may be such that the body weight/body composition meter 3 sends the body weight measurement values along with the measurement day/time to the server 5 in step S3. The configuration may be such that upon receiving the body weight measurement values and the measurement day/time from the body weight/body composition meter 3, the CPU 521 in the control unit 52 of the server 5 may obtain the measurement data 40 for the body weight measurement values, including the morning body weight and the evening body weight, and execute the processes according to the flowcharts in FIGS. 6 through 10, after which the data is stored in the storage unit 55, and the display is carried out in the display unit 54.

In this case, the server 5 may be configured so as to send the information displayed in the screen of the display unit 54 to the body weight/body composition meter 3. The body weight/body composition meter 3 may be configured to receive this information and display the received information in the display unit 15.

In this manner, even in the case where processing is carried out by the server 5, the measurement subject can be notified of the evening target body weight and the results of determining the degree of achievement. In addition, in the case where the server 5 is used in this manner, it is also possible for an instructor or the like to confirm the information and offer advice to a user.

The configuration may be such that the measurement subject makes an input by selecting a morning/evening button (not shown) in the operating unit 14 between step 51 and step S2. In this case, the morning body weight measurement and the evening body weight measurement can be distinguished based on the input. Accordingly, the body weight management device according to the present embodiment can also be used by a measurement subject whose sleeping and waking times are inverted, such as a shift worker.

In the case where this configuration is employed, the display content may be varied depending on whether or not the morning/evening button has been depressed, such as displaying only the body weight and not displaying targets or the like if the morning/evening button has not been depressed. Accordingly, the measurement subject can be prevented from forgetting to press the morning/evening button.

In addition, the display screens are not limited to the display screens shown in FIGS. 12A through 12F. Although the evening target body weight is displayed in the target display 63 in FIG. 12A, the configuration may be such that a difference between the evening target body weight and the morning body weight is displayed as a target value.

Furthermore, although the screens shown in FIGS. 12C and 12D display whether or not a target has been achieved using the difference display 83 and the target achievement result display 84, the display is not limited thereto; other appropriate displays may be carried out, such as displaying whether or not a target line has been exceeded using a graph display, or displaying one of a target achieved mark and a target not achieved mark.

In addition, the display timing is not limited to displaying both the morning body weight and the evening target body weight at the same time and displaying the evening measured body weight and the target achievement result at the same time; the displays may be carried out at various timings. For example, the configuration may be such that the measured body weight is displayed when the body weight measurement is complete, after which the display switches to the target display, the target achievement result display, or the like at a timing such as when a predetermined amount of time has passed following the measured body weight display, after the user has stepped off of the upper surface cover unit 35, or the like. Even in such a case, the diurnal target (evening target body weight) can be confirmed during the morning measurement, and the result of whether or not the diurnal target has been achieved can be confirmed.

In addition, although the aforementioned embodiment describes the basal metabolism amount being calculated based on the measurement subject's body impedance as detected by the impedance detection unit 20, the calculated body composition information is not limited to the basal metabolism amount. For example, a body fat percentage, BMI, visceral fat level, skeletal muscle percentage, body age, and so on may be calculated based on the body impedance, the height, age, and sex of the measurement subject stored in the storage unit 12, and the body weight detected by the load detection unit 33, and that calculated information may be outputted along with the body weight.

Furthermore, the stated body weight management method carried out by the body weight/body composition meter 3 according to the present embodiment can also be provided as a program. This program can also be recorded on a computer-readable recording medium 56, such as a flexible disk provided to the computer of the control unit 18 or the control unit 52, a CD-ROM (compact disk read-only memory), a ROM, a RAM, a memory card, and so on, and can then be provided as a program product. Alternatively, the program can be recorded on a recording medium such as a hard disk mounted within a computer, and can be provided in such form as a program. Further still, the program can also be downloaded via a network, and can be provided in such form as a program.

The provided program product is installed in a program storage unit such as a hard disk or the like and is then read out and executed by the CPU 181 (or 521). Note that the program product includes the program itself and the recording medium on which the program is recorded.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### REFERENCE NUMERALS LIST

1 body weight management system

3 body weight/body composition meter

5 server

10 display/operating unit

11, 51 communication unit

12, 55 storage unit

13 timer unit

14, 31, 53 operating unit

15, 54 display unit

16 constant current circuit unit

17 power source unit

18, 52 control unit

19 double integral AD unit

20 impedance detection unit

21, 36 electrode unit

30 body weight measurement unit

32 battery

33 load detection unit

34 load cell

35 upper surface cover unit

40 measurement data

41 standard diurnal fluctuation amount data

42 target setting day data

43 long-term target increase/decrease amount data

44 target achievement period data

45 initial body weight data

182 target obtainment unit

183 computation unit

184 target achievement determination unit

185 output processing unit

186 body weight calculation unit

187 increase/decrease amount calculation unit

1. A body weight management device comprising:
a body weight/body composition meter that obtains measurement data in which a body weight measurement value for a measurement subject and a measurement day/time are associated with each other;
a storage unit that stores said measurement data obtained by said body weight/body composition meter;
a target obtainment unit that, based on said measurement data in said storage unit, calculates a target diurnal fluctuation amount including a body weight fluctuation amount during sleep and a body weight diurnal target increase/decrease amount; and
a computation unit that, based on the target diurnal fluctuation amount obtained by said target obtainment unit, calculates a target value to serve as a target to be achieved when measuring a pre-sleep body weight value at a second timing near when the measurement subject sleeps, based on a post-waking body weight value measured at a first timing after the measurement subject wakes,
wherein said target obtainment unit calculates said diurnal target increase/decrease amount based on said measurement data in said storage unit so that said diurnal target increase/decrease amount is within a predetermined increase/decrease percentage range of said post-waking body weight value.

2. The body weight management device according to claim 1,
wherein said target obtainment unit compares a predetermined increase/decrease percentage in said predetermined increase/decrease percentage range from an initial body weight value at an initial day/time to a target body weight value set as a target to be achieved when body weight management is completed at a target date/time that serves as a day/time for completing the body weight management with a body weight increase/decrease percentage from a first day/time at which said measurement data was stored in said storage unit to a second day/time that is after said first day/time, and calculates said diurnal target increase/decrease amount based on a result of the comparison.

3. The body weight management device according to claim 1, wherein said target obtainment unit comprises:
a body weight calculation unit that calculates a predetermined body weight value at a predetermined day/time in a case where the body weight value changes in accordance with the predetermined increase/decrease percentage in said predetermined increase/decrease percentage range, from an initial body weight value at an initial day/time to a target body weight value at a target date/time; and
an increase/decrease amount calculation unit that compares said post-waking body weight value measured at a predetermined day/time with said predetermined body weight value calculated by said body weight calculation unit, and calculates said diurnal target increase/decrease amount based on a result of a comparison of the values.

4. The body weight management device according to claim 3,
wherein when the result of the comparison of said values indicates that said post-waking body weight value measured at said predetermined day/time is greater than said predetermined body weight value, said increase/decrease amount calculation unit calculates, as said diurnal target increase/decrease amount, the lesser of an increase/decrease amount corresponding to a predetermined percentage of said post-waking body weight value measured at said predetermined day/time and an increase/decrease amount according to said predetermined increase/decrease percentage; and
said predetermined percentage is a maximum increase/decrease percentage in said predetermined increase/decrease percentage range.

5. The body weight management device according to claim 3,
wherein when the result of the comparison of said values indicates that said post-waking body weight value measured at said predetermined day/time is less than said predetermined body weight value, said increase/decrease amount calculation unit calculates, as said diurnal target increase/decrease amount, the increase/decrease amount according to said predetermined increase/decrease percentage.

6. The body weight management device according to claim 3,
wherein when the result of the comparison of said values indicates that said post-waking body weight value measured at said predetermined day/time is equal to said predetermined body weight value, said increase/decrease amount calculation unit calculates said diurnal target increase/decrease amount by dividing a difference between said post-waking body weight value measured at said predetermined day/time and said target body weight value by a number of days from said predetermined day/time to said target date/time.

7. The body weight management device according to claim 1,
wherein said target obtainment unit obtains said body weight fluctuation amount during sleep by calculating, based on said measurement data, an average of a plurality of body weight fluctuation amounts during sleep, the plurality of body weight fluctuation amounts being differences between pre-sleep body weight values for a plurality of past days and post-waking body weight values for the next days.

8. The body weight management device according to claim 1, wherein said computation unit:
calculates, as a standard fluctuation amount, an average of a plurality of body weight fluctuation amounts during sleep, the plurality of body weight fluctuation amounts being differences between pre-sleep body weight values measured on a plurality of past days and post-waking body weight values for the next days, based on said measurement data;
divides said standard fluctuation amount by a difference between an average time of the measure day/times of the post-waking body weight values measured for a plurality of past days and a measurement time of the post-waking body weight value measured at said predetermined day/time based on said measurement data and corrects said post-waking body weight value using a value of the result of the division; and
calculates said target value using said corrected post-waking body weight value.

9. The body weight management device according to claim 1, further comprising:
an impedance detection unit that calculates a basal metabolism amount based on body information regarding the measurement subject's body and a body impedance,
wherein said target obtainment unit calculates said body weight fluctuation amount during sleep using said basal metabolism amount.

10. The body weight management device according to claim 1, further comprising:
an output processing unit that outputs said target value calculated by said computation unit.

11. The body weight management device according to claim 1, further comprising:
a target achievement determination unit that determines whether or not a diurnal target has been achieved using said pre-sleep body weight value and said post-waking body weight value; and
an output processing unit that outputs a result of the determination performed by said target achievement determination unit.

12. A body weight management method that manages a measurement subject's body weight using a computer, the method comprising:
a step of obtaining measurement data in which a body weight measurement value measured for the measurement subject and a measurement day/time are associated with each other;
a step of storing said measurement data in a memory;
a step of calculating, based on said measurement data in said memory, a target diurnal fluctuation amount including a body weight fluctuation amount during sleep and a body weight diurnal target increase/decrease amount; and
a step of calculating, based on said target diurnal fluctuation amount calculated in the step of calculating said target diurnal fluctuation amount, a target value to serve as a target when measuring a pre-sleep body weight value at a second timing before the measurement subject sleeps relative to a post-waking body weight value that is measured at a first timing after the measurement subject wakes,
wherein the step of calculating said target diurnal fluctuation amount includes comprises a step of calculating said diurnal target increase/decrease amount based on said measurement data in said memory so that said diurnal target increase/decrease amount is within a predetermined increase/decrease percentage range of said post-waking body weight value.

13. A non-transitory computer readable medium having a program stored thereon that causes a computer to execute a body weight management method for a measurement subject, said body weight management method comprising:
a step of obtaining measurement data in which a body weight measurement value measured for the measurement subject and a measurement day/time are associated with each other;
a step of storing said obtained measurement data in a memory;
a step of calculating, based on said measurement data in said memory, a target diurnal fluctuation amount comprising a body weight fluctuation amount during sleep and a body weight diurnal target increase/decrease amount; and
a step of calculating, based on said target diurnal fluctuation amount calculated in the step of calculating said target diurnal fluctuation amount, a target value to serve as a target when measuring a pre-sleep body weight value at a second timing before the measurement subject sleeps relative to a post-waking body weight value that is measured at a first timing after the measurement subject wakes,
wherein the step of calculating said target diurnal fluctuation amount comprises a step of calculating said diurnal target increase/decrease amount based on said measurement data in said memory so that said diurnal target increase/decrease amount is within a predetermined increase/decrease percentage range of said post-waking body weight value.

14. (canceled)
